(19) United States
(12) Patent Application Publication
(10) Pub. No.: US 2013/0243861 A1
(43) Pub. Date: Sep. 19, 2013

(54) PRESS-COATED TABLETS OF PREDNISONE

(71) Applicant: CADILA HEALTHCARE LIMITED, Ahmedabad (IN)

(72) Inventors: Sunilendu Bhushan ROY, Dist. Ahmedabad (IN); Sushrut Krishnaji KULKARNI, Dist. Ahmedabad (IN); Ajaykumar HANDA, Dist. Ahmedabad (IN); Ketan Tulsidas SAVJANI, Dist. Ahmedabad (IN)

(73) Assignee: CADILA HEALTHCARE LIMITED, Ahmedabad (IN)

(21) Appl. No.: 13/803,964

(22) Filed: Mar. 14, 2013

(30) Foreign Application Priority Data
Mar. 16, 2012 (IN) 697/MUM/2012

(51) Int. Cl. A61K 9/28; A61K 31/573

(52) U.S. Cl. CPC A61K 9/2866; A61K 31/573; A61K 9/284
USPC 424/480; 424/474; 514/178; 424/482

(57) ABSTRACT

The present invention provides for press-coated tablets of prednisone comprising a core comprising prednisone and a coating around the core. The present invention particularly discloses thickness of the coating applied to core having a convex shape for chronotherapeutic use. The present invention also provides for a process for preparing a press-coated tablet of prednisone and a method for treating conditions or pathology, the symptoms of which occur early in the morning.

### FIELD OF INVENTION

The present invention relates to press-coated tablets of prednisone comprising a core comprising prednisone and a coating around the core. The present invention specifically relates to thickness of coating applied to core having a convex shape for chronotherapeutic use. Moreover, the present invention also relates to a process for preparing a press-coated tablet of prednisone and a method for treating conditions or pathology, the symptoms of which occur early in the morning.

### BACKGROUND OF THE INVENTION

Prednisone is a non-flourinated synthetic corticosteroid drug and is used in the treatment of inflammatory disease. The chemical name for prednisone is pregna-1,4-diene-3,11,20-trione, 17,21-dihydroxy- and its molecular weight is 358.43. The structural formula is represented below:

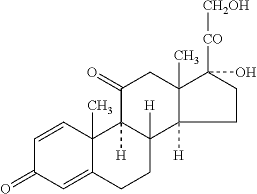

Prednisone is generally recommended in the treatment of immunoinflammatory disorders like rheumatoid arthritis, osteoarthritis, psoriasis, ulcerative colitis, crohn's disease, stroke-induced brain cell death, ankylosing spondylitis, fibromyalgia, and autoimmune diseases such as asthma, multiple sclerosis, type I diabetes, systemic lupus erythematosus, scleroderma, systemic sclerosis, and Sjogren's syndrome. Prednisone is white, odourless and very slightly soluble in water and is available in the market in conventional immediate release tablet dosage form. However, there are no dosage forms available in the market for the treatment of night time disease or early morning pathology.

In particular, if symptoms of a disease become apparent at night, or in the early hours upon waking, the time when a patient must take its medication in order to affect the best clinical outcome requires detailed consideration. For example, most asthma attacks occur in the early hours of the morning, e.g. 4 am to 6 am. This is a result of complex circadian rhythms such as the secretion of hydrocortisone and adrenaline. Ischaemic heart diseases occur most often during the night or in the early waking hours around breakfast time. Stiffness and pain associated with rheumatoid arthritis and osteoarthritis occur in the early waking hours, which is believed to be as a result of the secretion of IL-6 in the early hours of the morning, e.g. around 2 am to 4 am.

With conventional immediate release dosage forms, synchronization of drug administration with a nocturnal circadian rhythm responsible for the symptoms experienced by a patient would require a patient having to be disturbed from sleep to take a medicament during the early hours of the morning in order to achieve the most efficacious clinical outcome. Of course, this would be highly inconvenient for a patient.

Currently, numerous researches are going for the treatment of circadian rhythm disorders and in chronotherapeutic field, which have demonstrated the importance of biological rhythms in drug therapy. The optimal clinical outcomes cannot be achieved if a drug is released constantly after ingestion. In such cases, drug release should vary in a manner that is sympathetic to these variations in order that drug plasma concentrations are at an optimal therapeutic level only when required to treat symptoms of a diseased state.

Accordingly, there remains a need to provide dosage forms that can be taken at a convenient hour before bedtime that will release an effective dose of a drug substance only after a pre-determined lag time in order to synchronise peak plasma concentrations of drug with a particular circadian rhythm.

Furthermore, particularly in relation to drug substances that have a narrow absorption window, or in the case of drug substances that are adapted to treat a local condition in the colon such as Crohn's disease, ulcerative colitis, IBS and IBD, there is also a need to provide a dosage form that rapidly releases the drug substances after reaching the end of the lag time.

Still further, having regard to the varied life styles of patients, in order to reduce the inter- and intra-subject variance in bioavailability, there is a need to provide a dosage form that releases a drug with a reliable lag time, and to provide peak plasma drug concentrations at a pre-determined time, irrespective of whether a patient is in a fed or fasted state.

PCT Application Number WO 2002/072033 discloses the time controlled release dosage form of corticosteroids with a defined release rate. This dosage form is characterized by a coating containing a natural or synthetic gum that gels in the presence of aqueous media. The coating acts as a barrier to the ingress of aqueous media into an active-agent-containing core and thereby creating a lag time during which no drug substance is released. However, the problems associated with such type of dosage form are that release of the drug occurs by means of diffusion through the gelled coating. Second, to control lag time by controlling the coating weight remains uncertain to dosage form because increasing in coating weight adds additional cost to dosage form and increases the size of dosage form, which is difficult to swallow for patient. Also if the core is not positioned correctly in the die of the press coating machine, part of the coating may be intentionally thinner than the desired, which may cause premature drug release.

U.S. Pat. No. 6,183,780 discloses the oral delayed immediate release formulation comprising a compressed core containing one or more active substances surrounded with a coating, wherein release of active substance from the core is caused by rupture of the coating after a definite lag-time, said core comprising one or more immediate release carriers and having no substantial swelling properties upon exposure to gastrointestinal fluids.

US Application Number 2007/0110807 discloses the press-coating tablet of corticosteroids comprising a core with specified geometry and coating around said core such that the core is being disposed correctly within the coating. It discloses the press-coated tablet of corticosteroids having a coating thickness about an axis (X-Y) is greater than about an axis (A-B) orthogonal to (X-Y).

Press-coated tablets may have certain drawbacks because, for example, the core must be placed precisely in the center of the die. A core that is off slightly to one side or the other could result in the uncontrolled delivery of the active agent. Thus, the inventors of the present invention have found that by locating the position of core geometrically in center of the coating, it is possible to achieve all or substantially all release of prednisone after the desired lag period.

The inventors of the present invention have surprisingly found that by carefully selecting the geometry of the core within its coating, it is possible to manipulate the coating thickness at specific points on the tablet which releases the drug with desired rate after specific lag time. The inventors of the present invention have found that the press-coated tablets of prednisone having a coating thickness about an axis (A-B) is equal to or thicker than about an axis (X-Y) orthogonal to axis (A-B), which will release the drug after desired lag time and can be used effectively in the treatment of early morning pathology.

### SUMMARY OF THE INVENTION

The present invention provides press-coated tablet comprising a core comprising prednisone and a coating around said core, wherein said core is geometrically located within the coating and said tablet exhibits delayed release pattern.

The present invention also provides for a press-coated tablet comprising a core comprising prednisone and a coating around said core, the core being disposed within said coating such that the coating thickness about an axis (A-B) is equal to or thicker than the coating about an axis (X-Y) orthogonal to axis (A-B).

The present invention also provides for a press-coated tablet comprising a core comprising prednisone and a coating around said core, the core being disposed within said coating such that the coating thickness about an axis (A-B) is equal to or thicker than the coating about an axis (X-Y) orthogonal to axis (A-B), and wherein the thickness of the coating about the axis (A-B) is such that the coating is adapted to rupture upon immersion in an aqueous medium after a period of between about 2 to about 6 hours.

The present invention also provides for a press-coated tablet comprising a core comprising prednisone and a coating around the core, wherein said tablet releases more than 80% of prednisone within 3 to 7 hours of administration.

The present invention also provides for a press-coated tablet comprising a core comprising prednisone, a one or more pharmaceutically acceptable excipients selected from the group consisting of diluents, fillers, binders, disintegrants, lubricants, glidants, colorants, or combination thereof and a coating around said core.

The present invention also provides for a press-coated tablet comprising a core comprising prednisone and swellable adjuvants, a coating around said core, wherein said tablet exhibits delayed release pattern.

The present invention also provides for a press-coated tablet comprising a core comprising prednisone and a coating around said core, wherein said coating comprising a hydrophilic or hydrophobic material.

The present invention further provides for a press-coated tablet comprising a core comprising prednisone and a coating around said core, the core being disposed within said coating such that the coating thickness about an axis (A-B) is equal to or thicker than the coating about an axis (X-Y) orthogonal to axis (A-B), wherein the composition exhibits no significant difference in both rate and extent of absorption of prednisone or salt thereof as compared to delayed release composition of prednisone marketed under trade name Rayos®.

The present invention also provides for a process for preparing a press-coated tablet of prednisone comprising the steps of: a) forming a core comprising prednisone and one or more pharmaceutically acceptable excipients; b) forming a mixture containing a coating material and; c) press-coating the mixture containing the coating material around the core.

The present invention provides for a method of treatment of arthritic pain, joints pain, allergies, asthma, crohn's disease, ulcerative colitis, irritable bowel syndrome, and inflammatory bowel disease of the upper and lower sections of intestines using a press-coated tablet comprising core comprising prednisone and a coating around said core.

### DETAILED DESCRIPTION OF THE DRAWING

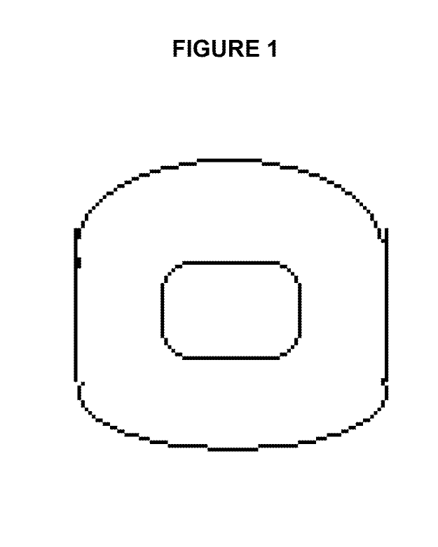

FIG. 1: represents the vertical section of press-coated tablet showing the axis (A-B) and the axis (X-Y) orthogonal to axis (A-B). Here, (A-B) axis is the direction of the movement of punches.

### DETAILED DESCRIPTION OF THE INVENTION

The inventors of the present invention have invented that press-coated tablet of prednisone having a core located geometrically within the specific coating is able to provide the control on lag time and all or substantially all the drug is rapidly released after the completion of the lag time and at the intended absorption site of the GI tract.

Accordingly, there is provided a press-coated tablet comprising a core containing prednisone and a coating around said core, such that coating is applied using a compression process and said tablet exhibits delayed release profile of prednisone.

The term “prednisone” used throughout the specification refers to not only prednisone per se, but also its pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs and pharmaceutically acceptable prodrugs thereof.

The inventors of the present invention have established that press-coated tablet comprising a core containing prednisone and a coating around said core, the core is being disposed within the coating such that the coating thickness about the axis (A-B) is equal to or thicker than the coating thickness about the axis (X-Y) orthogonal to axis (A-B) and wherein said coating provides a lag time between about 2 to about 6 hours followed by substantially complete release of the drug.

The press-coated tablet of the present invention comprises a core comprising prednisone and a coating around said core, wherein said tablet release more than 80% of prednisone within 3 to 7 hours of administration, preferably within 4 hours of administration.

The delayed release composition of prednisone or salt thereof in accordance with present invention was also found to exhibit no significant difference in both rate and extent of absorption of prednisone or salt thereof as compared to delayed release composition of prednisone marketed under trade name Rayos®.

In one embodiment of the present invention, a press-coated tablet comprises a core comprising prednisone and a coating around said core, the core being disposed within the coating such that the thickness about the axis (A-B) is between 2.1 to about 3.0 mm.

In another embodiment of the present invention, a press-coated tablet comprises a core comprising prednisone and a coating around said core, the core being disposed within the coating such that the thickness about the axis (X-Y) is between about 1.0 to 2.1 mm.

According to the present invention, the thickness of the coating should not be so thick that it renders the final formulation too large to be swallowed by the patients or it should not be so thin that it renders the final formulation very weak and brittle which breaks during the mechanical handling. The thickness of the coating about the axis (X-Y) is between about 1.0 to 2.1 mm. The coating thickness of the either side of the core on the axis (X-Y) may or may not be equal. For example, on a first side of the core (X-core), the coating may have a thickness of about 1.6 to about 1.8 mm, while on the other side (Y-core) of the coating may have a thickness of about 1.9 mm to about 2.0 mm.

In another embodiment of the present invention, a press-coated tablet comprises a core comprising prednisone and a coating around said core, the core being disposed within the coating such that the thickness about the axis (A-B) is at least 2.1 mm, preferably 2.1 to about 3.0 mm, and the thickness about the axis (X-Y) orthogonal to axis (A-B) is between about 1.0 to 2.1 mm.

In yet another embodiment of the present invention, a press-coated tablet comprises a core comprising prednisone and a coating around said core, such that the coating thickness about the axis (A-B) is equal to or thicker than the coating thickness about the axis (X-Y). The ratio of the thickness of the coating about the axis (A-B) to the thickness of the coating about the axis (X-Y) may vary from about 1:1 to about 3:1.

The thickness of the coating along and about the axis of the direction of movement of the punch (the “(A-B)” axis referred to above) is determined by the amount of coating material added to the die and the compaction force applied to form the tablet. On the other hand, the thickness of the coating along and about the “(X-Y)” axis is determined by the size of the core, its position within the die and the diameter of the die.

The hardness of the tablet may preferably be at least 60 Newtons, e.g. 60 to 80 Newtons, and more particularly 60 to 75 Newtons. Hardness may be measured according to a process described in The European Pharmacopoeia 4, 2.9.8 at page 201. The test employs apparatus consisting of 2 opposing jaws, one of which moves towards the other. The flat surfaces of the jaws are perpendicular to the direction of movement. The crushing surfaces of the jaws are flat and larger than the zone of contact with the tablet. The apparatus is calibrated using a system with a precision of one Newton. The tablet is placed between the jaws. For each measurement, the tablet is oriented in the same way with respect to the direction of the applied force. Measurements are carried out on 10 tablets. Results are expressed in terms of the mean, minimum and maximum values (in Newtons) of the force needed to crush the tablets.

In the present invention, tablets having core located geometrically within the coating may be adapted to release the prednisone after a pre-determined lag time. The prednisone may be released within short period of time after the completion of the lag time, more particularly all or substantially all the prednisone may be released within short span after the pre-determined lag time. This ensures that prednisone may be released at the intended absorption site along the GI tract within about ½ hour or about 1 hour after the expiry of the lag period.

According to the present invention, it is important to analyse the effect of food on the absorption of drug from the GI tract. It is well known that the rate at which a tablet will pass through the GI tract will vary depending on whether a patient is in a fed or fasted state. In the fasted state, a tablet will typically clear the stomach within about ½ hour and 1 hour after ingestion, and thereafter take a further 4 to 5 hours to clear the upper GI tract through the ileosecal junction. In a fed state, a tablet may take as long as 4 hours to be cleared from the stomach, and a further 4 to 5 hours to clear the upper GI tract. Accordingly, if a tablet is to release of all, or substantially all, of its drug into the upper GI tract irrespective of the fed state of a patient, it is preferable that the release of the drug after the lag time occurs within a time limit referred to in the paragraph above.

It is important to consider that whereas it is desirable that no drug substance is released during the lag time, some release may occur. However, any release of drug substance during the lag time should not exceed 10% of the total amount of drug substance in the core.

In another embodiment of the present invention, a press-coated tablet comprises a core comprising prednisone and one or more pharmaceutically acceptable excipients selected from the group consisting of diluents, fillers, binders, swellable adjuvants, lubricants, glidants, colorants and a coating around said core.

The amount of prednisone employed in tablets of the present invention will depend on the condition of the patient and the nature and the severity of the condition to be treated. Preferably, prednisone may be present from about 1 to about 50% by weight of the core, more preferably from about 5 to 30% by weight of the core.

Suitable diluents or fillers are selected from, but are not limited to microcrystalline cellulose, starch, dibasic calcium phosphate, tribasic calcium phosphate, calcium carbonate, dextrose, kaolin, magnesium carbonate, magnesium oxide; sugars such as lactose or sucrose; sugar alcohols such as mannitol, sorbitol, erythritol and the like. The diluents or fillers may present in an amount from about 10% to about 90% by weight of the core, preferably from about 30% to about 70% by weight of the core, more preferably from about 20% to about 50% by weight of the core.

Suitable binders are selected from, but are not limited to hydroxyethyl cellulose, hydroxypropyl cellulose, hydroxypropyl methylcellulose, carbomers, dextrin, ethyl cellulose, methylcellulose, shellac, zein, gelatin, polymethacrylates, polyvinyl pyrrolidone, pregelatinized starch, sodium alginate, gums, synthetic resins or combination thereof. The binders may present in amount from about 1% to about 25% by weight of the core, preferably from about 10% to about 20% by weight, more preferably from about 5% to about 10% by weight of the core.

Suitable swellable adjuvants are selected from, but are not limited to sodium carboxymethyl cellulose, crosslinked polyvinylpyrrolidone, sodium alginate, sodium starch glycolate and combination thereof. The swellable adjuvants may present in amount from about 1% to about 25% by weight of the core, preferably from about 10% to about 20% by weight, more preferably from about 5% to about 10% by weight of the core.

Suitable lubricants and glidants are selected from, but are not limited to talc, metallic stearates such as magnesium stearate, calcium stearate, zinc stearate; colloidal silicon dioxide, finely divided silicon dioxide, stearic acid, hydrogenated vegetable oil, glyceryl palmitostearate, glyceryl monostearate, glyceryl behenate, polyethylene glycols, powdered cellulose, starch, sodium stearyl fumarate, sodium benzoate, mineral oil, magnesium trisilicate, kaolin; or combination thereof. The lubricants and glidants may present in amount from about 1% to 25% by weight of the core, preferably from about 5% to about 20% by weight of the core, more preferably from about 2% to about 10% by weight of the core.

Suitable examples of colorants are selected from, but are not limited to non-water soluble lake pigments; neutral pigments; yellow ferric oxide; red ferric oxide; black iron oxide and the like. The colorants may present in amount from about 1% to about 10% by weight of the core, preferably from about 1% to about 5% by weight of the core, more preferably from about 2% to about 4% by weight of the core.

In another embodiment of the present invention, a press-coated tablet comprises a core containing prednisone and a coating around said core, wherein said coating comprises a hydrophilic or a hydrophobic rate controlling material alone or in combination in the composition.

In use, the coating optimally acts merely as a barrier to the ingress of aqueous physiological media thereby providing a drug release lag time. For the reasons set forth above, optimally the tablet should have the minimum thickness possible consistent with the desired lag time.

Suitable hydrophilic rate controlling materials are selected from, but are not limited to alkyl celluloses such as methyl cellulose; hydroxyalkyl celluloses, for example, hydroxymethyl cellulose, hydroxyethyl cellulose, hydroxypropyl cellulose, and hydroxybutyl cellulose; hydroxyalkyl alkyl celluloses such as hydroxyethyl methyl cellulose and hydroxypropyl methyl cellulose; carboxyalkyl cellulose or esters; microcrystalline cellulose, crosslinked cellulose derivatives such as crosslinked sodium carboxymethyl cellulose; crosslinked polyvinyl pyrrolidone and vinyl acetate (commercially available grade such as Kollidon VA64); polysaccharides such as galactomannans, tragacanth, agar, guar gum, and polyfructans; polyvinyl alcohol; polyethylene glycol, polyvinylpyrrolidone, copolymers of polyvinylpyrrolidone with vinyl acetate; combinations of polyvinyl alcohol and polyvinylpyrrolidone; and polyalkylene oxides such as polyethylene oxide and polypropylene oxide and copolymers of ethylene oxide and propylene oxide, preferably cellulose ether derivatives such as hydroxypropyl methyl cellulose and hydroxypropyl cellulose, most preferred hydroxypropyl methyl cellulose and the like. Preferred rate controlling polymer is hydroxypropyl methyl cellulose. The hydrophilic rate controlling materials may present in an amount from about 5% to about 85% by weight of the coating materials, preferably from about 10% to about 70% by weight of the coating materials, more preferably from about 30% to about 50% by weight of the coating materials.

Different viscosity grades of hydroxypropyl cellulose and hydroxypropyl methyl cellulose are commercially available. Hydroxypropyl methyl cellulose (HPMC) preferably used in the present invention has a viscosity grade ranging from about 3,500 mPas to about 1,00,000 mPas, in particular ranging from about 4,000 mPas to about 20,000 mPas and most in particular a viscosity grade of about 6,500 mPas to about 15,000 mPas (apparent viscosity of a 2% aqueous solution at 20° C.), e.g., hypromellose 2208 or 2206 (DOW, Antwerp, Belgium). HPMC type 2208 contains 19-24% by weight methoxy and 4-12% by weight hydroxypropoxy substituents.

Suitable hydrophobic rate controlling materials for coating are selected from, but are not limited to one or more of glyceride (e.g., glyceryl behenate, glyceryl trimyristate, glyceryl trilaurate, glyceryl tristearate, glyceryl monostearate, glyceryl palmitostearate, or glyceryl triacetate), stearic acid, hydrogenated castor oil, a hydrogenated vegetable oil, a water insoluble cellulose (e.g., ethyl cellulose, cellulose acetate, cellulose acylate, cellulose diacylate, cellulose triacylate, cellulose acetate butyrate, cellulose acetate propionate, nitrocellulose, cellulose diacetate, or cellulose triacetate), a wax or a wax-like substance (e.g., carnauba wax, cetyl esters wax, beeswax, castor wax, cationic emulsifying wax, cetrimide emulsifying wax, an emulsifying wax, microcrystalline wax, a nonionic wax, a nonionic emulsifying wax, paraffin, petroleum wax, petroleum ceresin wax, spermaceti wax, white wax, or yellow wax), a fat, an oil, a fatty acid, an emulsifier, a modified starch, a fatty alcohol, a protein (e.g., zein), shellac, or a polymer (e.g., a polyolefin, a polyurethane, a polyvinylchloride, a polyvinyl acetate, an acrylic acid polymer, a methacrylic acid polymer); cetostearyl alcohol, stearyl alcohol; and the like.

The coating composition may optionally include fillers, such as water-soluble diluents or fillers, water-insoluble diluents or fillers, and mixtures thereof. Preferably, the filler is water-soluble. Exemplary water soluble fillers include a water-soluble sugar, lactose, glucose, fructose, sucrose, galactose, mannose, the corresponding sugar alcohols and other sugar alcohols, such as mannitol, sorbitol, and xylitol. Most preferably, the water soluble filler is lactose or granulated mannitol. The water-insoluble fillers can include, for example, calcium salt or talc. A preferred insoluble calcium salt is a calcium phosphate, more preferably dicalcium phosphate. The filler in the coating composition may be the same or different as the filler in the core composition, if any. For example, the core composition may include a water-soluble filler while the coating composition may include a water-insoluble filler. In a preferred embodiment, the filler material is the same in both the core and the coating compositions.

The coating composition may optionally include other excipients, such as binders, lubricants, processing aids, pH buffers, glidants, colorants, and the like, which can be the same or different as those in the core composition, if any.

In still another embodiment, a press-coated tablet may be further coated with optional additional coatings. The additional coatings may be aesthetic or functional, and may include the active ingredient. Such additional coatings preferably include film forming materials.

In a preferred embodiment of the invention, the additional coating may be aesthetic coating. In another preferred embodiment, the additional coating may include an active ingredient, either the same or different as the active ingredient contained in the core and press-coat. Preferably, the active ingredient in the additional coating may desirably administer along with the active ingredient in the core and press-coat. In other preferred embodiments, the additional coating may include an immediate release loading dose of the same active ingredient that is found in the core and press-coat of the formulation.

The diameter of the core of tablet may range from about 5.0 mm to about 6.0 mm and the thickness of the core may range from about 1.5 mm to about 3.0 mm. The shape of the delayed release tablet of the present invention may depend on the shape and size of punches used. For example, the tablets of the present invention may be of convex shape.

The invention in further aspect relates to a process for preparing press-coated tablets. The tablets may be prepared by conventional press-coating equipment. Typically such equipment is composed of a series of die arranged on a rotating platform. The dies are removably mounted in the platform such that differently sized die may be employed as appropriate. Each die is hollow to receive a lower punch. The punch is positioned within the die such that the upper surface of the punch and the inner surface of the die define a volume for receiving a precise amount coating material. Once loaded, the platform is rotated until the die is positioned under an upper punch. The upper punch is then urged down onto the coating material under a defined compression force and the coating material is pre-compressed or tamped between the upper and lower punch. A pre-formed core is then fed into die to rest on the tamped coating. Conventional press-coating apparatus may be equipped with centering devices that enable cores to be positioned both vertically and radially. This might be achieved by a tamping process, whereby an initial amount of coating material is placed in a die and is tamped with a shaped punch, such as a pin punch, that leaves an indentation in the coating material in which to receive a core. Thereafter, in a second filling operation, a precise amount of coating material is fed into the die to cover the core, and an upper punch compresses the coating material with a defined compaction force to form tablets according to the present invention.

The compression force applied during the tamping process is relatively light and is just sufficient to provide a bed of coating material to receive the core and to prevent movement of the coating material as a result of centrifugal force. Subsequent compression to form the tablet may be adjusted to give tablets of requisite hardness.

The amount of coating material fed into the die can be precisely defined having regard to the density of the coating material to ensure after compression that the tablet is formed with the required coating thickness about the (A-B) axis; and the dimensions of the die is selected to provide the thickness about the (X-Y) axis.

The core of the press-coated tablet comprising prednisone may be prepared by processes known to the person having ordinary skill in the art of pharmaceutical technology such as direct compression, dry granulation, slugging, hot melt granulation, hot melt extrusion, fluidized bed granulation, extrusion-spheronization, spray drying and solvent evaporation.

The process for producing the tablet takes place under usual conditions of the pharmaceutical industry. Thus, standard technologies are used in the production of the core tablet, such as weighing, sieving, mixing, aqueous granulation in a high-speed mixer, fluidized-bed drying of the granules, mixing and compression. Comparable methods are employed to produce the coating, namely weighing, sieving, mixing, aqueous granulation in a high-speed mixer, fluidized-bed drying of the granules, mixing and compression to press-coated tablets.

The process for preparing a press-coated tablet of prednisone comprises the steps of: a) forming a core comprising prednisone and pharmaceutically acceptable excipients; b) forming a mixture containing a coating material and; c) press-coating the mixture of the coating material around the core.

In one embodiment, the process for preparing a press-coated tablet of prednisone comprising the steps of:

a) forming core granules comprising prednisone and a pharmaceutically acceptable excipients selected from diluents, binders, disintegrants, colorants in a suitable mixer;
b) mixing the lubricants and glidants extra-granularly into the core granules;
c) compressing the material of step b) into core tablets using suitable size punches;
d) forming granules of coating material using diluents, polymer, binder by wet granulation and mixing said granules with glidants, lubricant and colorant;
e) press-coating the granules of coating material around the tablets of step c).

In another embodiment, a process for preparing a press-coated tablet of prednisone comprising the steps of:

### 1) Preparation of Core Tablet:

a) mixing a core material comprising prednisone, lactose, croscarmellose sodium in a suitable mixer or blender;
b) dissolving povidone in isopropyl alcohol and preparing the granules using the mixture of step a);
c) adding croscarmellose sodium, colloidal silicone dioxide extra-granularly and at last lubricate the granules using magnesium stearate;
d) compressing the granules into a core tablet,

### 2) Preparation of Coating Material:

e) mixing hydroxypropylmethylcellulose (HPMC), dibasic calcium phosphate, povidone and colloidal silicon dioxide in a suitable mixer or blender;
f) lubricating the mixture of step e) using magnesium stearate for a prescribed time;
g) compressing the mixture of step e) on to the core tablet of step d).

The present invention further relates to a method of treatment of early morning pathology patients, in which the chronotherapeutic drug release is required. As already stated, the symptoms of these conditions appear according to a circadian rhythm and with great predictability during the early hours of the morning. Accordingly, prednisone is eminently suited for delivery from tablets according to this invention not only because of their narrow absorption window, but also because a tablet may be administered in the evening before bedtime anytime around 8 pm until midnight, e.g. around 10-12 at night, to deliver maximum plasma concentration of the drug substance before maximum secretion of IL-6 (which occur around 2 am to 4 am), thereby effectively addressing the underlying causes of the morning symptoms. In this way, these symptoms are more effectively treated.

Thus according to the present invention, a press-coated tablet of prednisone may be used in the method of treatment of arthritic pains like rheumatoid arthritis, osteoarthritis, joints pain, allergies, asthma, Crohn's disease, ulcerative colitis, irritable bowel syndrome, and inflammatory bowel disease of the upper and lower sections of intestines.

The bioequivalence studies were carried out between Rayos® delayed release tablets (reference) and compositions of the invention (test) in fed state. The study was monitored in terms of Cₘₐₓ and AUC achieved with the test product and the reference product (Rayos®).

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example 1

TABLE 1

| | | Quantity per Tablet (mg) | | |
|---|---|---|---|---|
| Sr. No. | Ingredients | 1 mg | 2 mg | 5 mg |

Core Tablet

| | | | | |
|---|---|---|---|---|
| 1 | Prednisone | 1.00 | 2.00 | 5.00 |
| 2 | Lactose | 42.8 | 41.8 | 38.8 |
| 3 | Croscarmellose Sodium | 4.00 | 4.00 | 4.00 |
| 4 | Povidone K-30 | 4.00 | 4.00 | 4.00 |
| 5 | Isopropyl Alcohol | q.s | q.s | q.s |
| 6 | Colloidal Silicon Dioxide | 0.30 | 0.30 | 0.30 |
| 7 | Croscarmellose Sodium | 7.00 | 7.00 | 7.00 |
| 8 | Magnesium Stearate | 0.60 | 0.60 | 0.60 |

Coating material

| | | | | |
|---|---|---|---|---|
| 9 | Hydroxypropylmethylcellulose | 50.00 | 50.00 | 50.00 |
| 10 | Dibasic Calcium Phosphate | 262.80 | 262.80 | 262.80 |

Core Tablet

| | | | | |
|---|---|---|---|---|
| 11 | Colloidal Silicon Dioxide | 3.00 | 3.00 | 3.00 |
| 12 | Povidone K-30 | 30.00 | 30.00 | 30.00 |
| 13 | Magnesium Stearate | 4.00 | 4.00 | 4.00 |
| | | 410.00 | 410.00 | 410.00 |

### Procedure:

### Preparation of Core Material:

Blend of Prednisone containing lactose, croscarmellose sodium was mixed in a suitable mixer or blender for a prescribed time. Then povidone K-30 was dissolved in a suitable solvent like IPA and was mixed with prednisone blend for granulation. The wet mass was dried in a suitable drying equipment to get the predetermined % Loss on drying. The dried mass of granules was passed through mesh or screen along with colloidal silicon dioxide. The sized granules were mixed with extra-granular material like croscarmellose sodium and were lubricated by magnesium stearate in a blender for a prescribed time. At last, the lubricated blend was compressed into tablets using suitable size punches.

### Preparation of Blend for Compression Coating

Blend of hydroxypropylmethylcellulose (HPMC), dibasic calcium phosphate, povidone K-30, and colloidal silicon dioxide were mixed in a suitable mixture or blender for a prescribed time and were lubricated with magnesium stearate for a prescribed time. Then, the coating materials were compressed around the tablets and were packaged in a specified primary packaging.

### Example 2

TABLE 2

| | | Quantity per Tablet (mg) | | |
|---|---|---|---|---|
| Sr. No. | Ingredients | 1 mg | 2 mg | 5 mg |

Core Tablet

| | | | | |
|---|---|---|---|---|
| 1 | Prednisone | 1.00 | 2.00 | 5.00 |
| 2 | Microcrystalline Cellulose | 42.8 | 41.8 | 38.8 |
| 3 | Crospovidone | 4.00 | 4.00 | 4.00 |
| 4 | Povidone K-30 | 4.00 | 4.00 | 4.00 |
| 5 | Water | q.s | q.s | q.s |
| 6 | Colloidal silicon dioxide | 0.30 | 0.30 | 0.30 |
| 7 | Croscarmellose Sodium | 7.00 | 7.00 | 7.00 |
| 8 | Sodium Stearyl Fumarate | 0.60 | 0.60 | 0.60 |

Coating material

| | | | | |
|---|---|---|---|---|
| 9 | Glyceryl Behenate | 100.00 | 100.00 | 100.00 |
| 10 | Dibasic Calcium Phosphate | 201.80 | 201.80 | 201.80 |
| 11 | Colloidal Silicon Dioxide | 3.00 | 3.00 | 3.00 |
| 12 | Povidone K-30 | 30.00 | 30.00 | 30.00 |
| 13 | Sodium Stearyl Fumarate | 5.00 | 5.00 | 5.00 |
| | | 410.00 | 410.00 | 410.00 |

### Procedure:

### Preparation of Core Material:

Blend of Prednisone containing microcrystalline cellulose, crospovidone was mixed in a suitable mixer or blender for a prescribed time. Then povidone K-30 was dissolved in a suitable solvent water and was mixed with prednisone blend for granulation. The wet mass was dried in a suitable drying equipment to get the predetermined % Loss on drying. The dried mass of granules was passed through mesh or screen along with colloidal silicon dioxide. The sized granules were mixed with extra-granular material croscarmellose sodium and were lubricated by sodium stearyl fumarate in a blender for a prescribed time. At last, the lubricated blend was compressed into tablets using suitable size punches.

### Preparation of Blend for Compression Coating

Blend of glyceryl behenate, dibasic calcium phosphate, povidone K-30, and colloidal silicon dioxide were mixed in a suitable mixture or blender for a prescribed time and were lubricated with sodium stearyl fumarate for a prescribed time. Then, the coating materials were compressed around the tablets and were packaged in a specified primary packaging.

### Example 3

TABLE 3

| | | Quantity per Tablet (mg) | | |
|---|---|---|---|---|
| Sr. No. | Ingredients | 1 mg | 2 mg | 5 mg |

Core Tablet

| | | | | |
|---|---|---|---|---|
| 1 | Prednisone | 1.00 | 2.00 | 5.00 |
| 2 | Lactose monohydrate | 42.7 | 41.7 | 38.7 |
| 3 | Croscarmellose Sodium | 4.0 | 4.0 | 4.0 |
| 4 | Povidone K-30 | 4.0 | 4.0 | 4.0 |
| 5 | Purified water | q.s. | q.s. | q.s. |
| 6 | Colloidal Silicon Dioxide | 0.3 | 0.3 | 0.3 |
| 7 | Croscarmellose Sodium | 7.0 | 7.0 | 7.0 |
| 8 | Sodium stearyl fumarate | 1.0 | 1.0 | 1.0 |

Coating

| | | | | |
|---|---|---|---|---|
| 9 | Hydroxypropylmethylcellulose | 40.0 | 40.0 | 40.0 |
| 10 | Lactose monohydrate | 202.0 | 202.0 | 202.0 |
| 11 | Microcrystalline cellulose | 110.0 | 110.0 | 110.0 |
| 12 | Povidone | 30.0 | 30.0 | 30.0 |
| 13 | Isopropyl alcohol | q.s. | q.s. | q.s. |
| 14 | Hydroxypropylmethylcellulose | 10.0 | 10.0 | 10.0 |
| 15 | Colloidal Silicon Dioxide | 3.0 | 3.0 | 3.0 |
| 16 | Sodium stearyl fumarate | 5.0 | 5.0 | 5.0 |
| | Total weight | 460.0 | 460.0 | 460.0 |

### Procedure:

### Preparation of Core Material:

Prednisone, lactose monohydrate, croscarmellose sodium were mixed in a suitable mixer or blender for a prescribed time. Povidone K-30 was dissolved in purified water to provide a binder solution. The blend of prednisone was granulated with the binder solution. The wet mass was dried in a suitable drying equipment to get the predetermined % Loss on drying. The dried mass of granules was passed through mesh or screen along with colloidal silicon dioxide. The sized granules were mixed with extra-granular material like croscarmellose sodium and sodium stearyl fumarate in a blender for a prescribed time. At last, the lubricated blend was compressed into tablets using suitable size punches.

### Preparation of Blend for Compression Coating:

Hydroxypropylmethylcellulose (HPMC), lactose monohydrate and microcrystalline cellulose were mixed together. The mixture was granulated with the binder solution containing povidone in IPA. The wet mass was dried in a suitable drying equipment to get the predetermined % Loss on drying. The dried mass of granules was passed through mesh or screen along with colloidal silicon dioxide. The sized granules were mixed with extra-granular material like hydroxypropylmethylcellulose and sodium stearyl fumarate in a blender for a prescribed time. The coating materials were compressed around the tablets and were packaged in a specified primary packaging.

TABLE 4
Dissolution profile of Example 3
(Conditions: Dissolution media - water in presence of 0.2% SLS,
500 ml, USP Type II apparatus (Paddle), 100 RPM)

| | % Drug release | | |
|---|---|---|---|
| Time (hr) | 1 mg | 2 mg | 5 mg |
| 1 | 0.0 | 0.0 | 0.0 |
| 2 | 0.0 | 0.0 | 0.0 |
| 4 | 0.0 | 0.0 | 0.0 |
| 6 | 82.3 | 92.5 | 85.1 |
| 8 | 102.9 | 96.5 | 100.4 |
| 10 | 103.5 | 97.3 | 101.3 |

### Example 4

In-vivo study was conducted in healthy human volunteers to assess bioavailability of Example 3—Prednisone delayed release tablets (5 mg) of the invention with that of Rayos® (Prednisone delayed release tablets, 5 mg)

TABLE 5

| | Ratio | CI_90 | CI_90 |
|---|---|---|---|
| Dependent | [% Ref] | Lower | Upper |
| Ln (Cₘₐₓ) | 113.66 | 101.09 | 127.80 |
| Ln (AUC₀₋ₜ) | 109.49 | 96.19 | 124.64 |

Table 5 provides summary of Pharmacokinetic parameters of delayed release formulations under Fed condition.

While the invention has been described in terms of its specific embodiments, certain modifications and equivalents will be apparent to those skilled in the art and are intended to be included within the scope of the invention.

1. A press-coated tablet comprising a core comprising prednisone, and a coating around the core, said core being disposed within said coating such that the coating thickness about an axis (A-B) is equal to or thicker than the coating thickness about an axis (X-Y) orthogonal to (A-B), wherein the axis (A-B) is the axis of the direction of movement of the punch used in the press-coating of the tablet.

2. The press-coated tablet according to claim 1, wherein the coating thickness about the axis (A-B) is between 2.1 mm to about 3.0 mm.

3. The press-coated tablet according to claim 1, wherein the coating thickness about the axis (X-Y) is between about 1.0 mm to 2.1 mm.

4. The press-coated tablet according to claim 1, wherein the tablet comprises a swellable core and a coating containing hydrophilic or hydrophobic material.

5. The press-coated tablet according to claim 4, wherein the core comprises a swellable adjuvant selected from sodium carboxymethyl cellulose, crosslinked polyvinylpyrrolidone, sodium alginate, sodium starch glycolate or combination thereof.

6. The press-coated tablet according to claim 1, wherein the coating comprises a hydrophilic material selected from hydroxypropyl cellulose, hydroxypropylmethylcellulose, polyvinylpyrrolidone, cross-linked polyacrylic acid, polyvinyl alcohol, carboxymethylcellulose, methyl cellulose, hydroxyethyl cellulose, starch derivatives, hydrocolloids including alginate, chitosan, pectin, poly (ethylene oxide), carbopol, gums or combination thereof.

7. The press-coated tablet according to claim 1, wherein the coating comprises a hydrophobic material selected from one or more of glyceride stearic acid, hydrogenated vegetable oils, a water insoluble cellulose, a wax or a wax-like substance, a fat, an oil, a fatty acid, an emulsifier, a modified starch, a fatty alcohol, a protein, shellac, or a polymer (e.g., a polyolefin, a polyurethane, a polyvinylchloride, a polyvinyl acetate, an acrylic acid polymer, a methacrylic acid polymer); cetostearyl alcohol, stearyl alcohol; or combination thereof.

8. The method of preparing a press-coated tablet according to claim 1, wherein the method comprises the steps of:
a) forming a core comprising prednisone and one or more pharmaceutically acceptable excipients;
b) forming a mixture comprising a coating material; and
c) press-coating the mixture of the coating material around the core.

9. The press-coated tablet according to claim 1, wherein the lag time before the release of prednisone or salts thereof is from about 2 to about 6 hours upon administration to the patients.

10. The press-coated tablet according to claim 1, wherein the tablets release more than 80% of prednisone within 4 hours of administration to the patients.

11. A method of treating arthritic pain, joints pain, allergies, asthma, crohn's disease, ulcerative colitis, irritable bowel syndrome, and inflammatory bowel disease of the upper and lower sections of intestines by providing to a patient in need of a treatment a press-coated tablet according to claim 1 comprising core comprising prednisone and a coating around said core.

12. The press-coated tablet according to claim 1, comprising 1 mg, 2 mg or 5 mg prednisone.

13. The press-coated tablet according to claim 1, wherein the tablet exhibits no significant difference in rate and/or extent of absorption of prednisone as compared to commercially marketed formulation of prednisone marketed under the trade name Rayos®.
